SEQUENCE LISTING (4 sequences)

SEQ ID NO: 1 - DNA, 21 bp, Artificial Sequence
  Primer
tggttttgcaggaggctatac

SEQ ID NO: 2 - DNA, 20 bp, Artificial Sequence
  Primer
gtagatggccgagcggtact

SEQ ID NO: 3 - DNA, 21 bp, Artificial Sequence
  Primer
gcgacagtccactcttcagtt

SEQ ID NO: 4 - DNA, 20 bp, Artificial Sequence
  Primer
cccaggtccatcaggaaaca

(19) United States
(12) Patent Application Publication
(10) Pub. No.: US 2010/0063139 A1
(43) Pub. Date: Mar. 11, 2010

(54) USE IN A COSMETIC COMPOSITION OF LIPOCHROMAN-6 TO ENHANCE THE RADIANCE OF THE COMPLEXION OF THE SKIN, ESPECIALLY THE SKIN OF THE FACE

(75) Inventors: CARINE NIZARD, IVRY SUR SEINE (FR); MARIELLE MOREAU, MARCQ (FR); EMMANUELLE NOBLESSE, DONNERY (FR); PHILIPPE SCHAEFFER, SAINT PIERRE DES CORPS (FR); BERTRAND FRIGUET, PARIS (FR); ISABELLE PETROPOULOS, BOISSY SAINT LEGER (FR); CEDRIC PICOT, ERMONT (FR); MARTINE PERICHON, CHELLES (FR)

Correspondence Address: WOODCOCK WASHBURN LLP, CIRA CENTRE, 12TH FLOOR, 2929 ARCH STREET, PHILADELPHIA, PA, 19104-2891, US

(73) Assignee: LVMH RECHERCHE, SAINT JEAN DE BRAYE (FR)

(21) Appl. No.: 12/533,159

(22) Filed: Jul. 31, 2009

(30) Foreign Application Priority Data
Aug. 1, 2008 (FR) 08 55371

(51) Int. Cl. A61K 8/49; A61Q 19/00

(52) U.S. Cl. 514/456

(57) ABSTRACT

The invention relates to the use, in a cosmetic composition intended for topical application to the skin, of lipochroman-6 as an active intended to enhance and/or restore the radiance of the complexion of the skin, especially the skin of the face.

The invention also relates to a method of cosmetic care of the skin, especially the skin of the face, intended to enhance and/or restore the radiance of the complexion, which comprises the application to the part of the skin in question of an effective amount of this cosmetic composition.

### CROSS REFERENCE TO RELATED APPLICATIONS

This application claims the benefit of French Patent Application No. 08 55371, filed Aug. 1, 2008, the entirety of which is incorporated herein.

### TECHNICAL FIELD

The present invention relates to the use in a cosmetic composition of lipochroman-6 for enhancing the radiance of the complexion of the skin, especially of the face.

### BACKGROUND

Lipochroman-6 is a compound of formula (I):

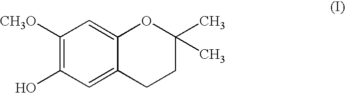

It is described in the prior art as an antioxidant, free-radical scavenger, as an inhibitor of lipid peroxidation and an agent for protecting cells against the damage induced by peroxynitrite. It is used as an anti-ageing agent in cosmetic compositions.

Reactive oxygen species (ROS) have the capacity to oxidize all of the cellular components, carbohydrates, DNA, lipids and proteins (Davies et al, J Biol Chem, 1987, 262(20): 9895-901; Halliwell et al, Am J Clin Nutr, 1993, 57(5 Suppl): 715S-24S; discussion 24S-25S; Stadtman, Methods Enzymol, 1995, 258: 379-93), leading to their degradation.

Consequently the cells have developed anti-oxidant defence mechanisms which are specific for the different oxidizing species, for the site where these species are produced and for their biological targets.

These mechanisms include on the one hand the non-enzymatic anti-oxidant defence, which employs compounds of low molecular weight such as melatonin, lipoic acid, coenzyme Q, melanin, vitamins C and E, and glutathione (GSH), and secondly the enzymatic anti-oxidant defence, such as superoxide dismutase (SOD), catalase, glutathione peroxidase, peroxiredoxins, sulfiredoxin, and the thioredoxin/thioredoxin reductase system.

Proteins are favoured targets of oxidative alterations. The oxidized proteins may be either broken down or repaired. One major system involved in the breakdown of oxidized proteins is the proteasome (Davies, Biochimie; 2001, 83(3-4): 301-10). One of the only systems for repairing oxidized proteins is the methionine sulphoxide reductase (Msr) system, which allows the process of oxidation to be reversed by reducing the methionine sulphoxides to methionines.

Methionine, an essential amino acid for the human species, constitutes an excellent agent for detoxification of ROS, also called a “trapping” or “scavenger” system, since it reacts very rapidly with a large part of the oxidizing species of the cell under physiological conditions (H₂O₂, OH., peroxinitrite, chloramine, hypochlorous acid (Levine et al, Proc Natl Acad Sci USA 1996, 93(26): 15036-40; Berlett et al, J Biol Chem, 1997, 272(33): 20313-6.; Tien et al, Proc Natl Acad Sci USA 1999, 96(14): 7809-14).

The majority of proteins contain at least one methionine in their primary sequence, and often the oxidation of these methionines gives rise to a loss of activity of the protein. An example is the protein which inhibits elastase activity at the level of the skin. This is because the oxidation of methionines in the primary sequence of amino acids of this inhibitory protein makes it ineffective at protecting the elastin.

The oxidative alterations of methionine within proteins may be repaired reversibly by the action of MsrA and MsrB, which are enzymes forming part of the Msr (methionine sulphoxide reductase) system.

The enzymes MsrA and MsrB (referred to collectively as Msr) are ubiquitous throughout the tissues of the body. Their role is to reverse the initial oxidation reaction of methionine to methionine sulphoxide (Met-S(O)), to form methionine again. This is one of the rare phenomena of reversible oxidation. The three-dimensional structures of their active sites are symmetrical. Each of the two enzymes specifically recognizes one diastereoisomer of methionine sulphoxide (S-form for MsrA and R-form for MsrB). Their role, although complementary, is therefore very specific.

Oxidized proteins have different optical properties from non-oxidized proteins. The technique of circular dichroism has shown that the light reflected by an oxidized protein is different from that reflected by a non-oxidized protein (Friguet et al, Arch Biochem Biophys, 1994, 311:168-73). Being more hydrophobic, they undergo aggregation more readily and may even crosslink with one another abnormally.

Apart from oxidized proteins, fluorescent compounds such as lipofuscin are also capable of accumulating in the cells (Brunk et al, Free Radic Biol Med, 2002, 33: 611-9).

Lipofuscin, a fluorescent pigment formed from the oxidation and aggregation of proteins and lipid residues, is now considered to be a marker of cell ageing (Terman et al, Int J Biochem Cell Biol, 2004, 36: 1400-4).

Consequently the accumulation of oxidized proteins and/or lipofuscin may have an impact on the skin, and more particularly on the complexion of the skin, by giving rise to dysregulation of the cell cycle at the level of the skin, resulting in a change in its natural, genetically determined colouring, and on its visual perception by an observer. The complexion is then said to be “dull”.

This alteration may be linked to the phenomenon of skin ageing, whether intrinsic, i.e. genetically determined, or extrinsic, i.e. caused by external factors such as UV. Its origin may also lie in disease, stress, tobacco consumption or any other factor able to induce oxidation of the proteins at the cellular level.

As such, methods for enhancing or restoring the radiance of the complexion of the skin of the face and/or body are needed.

### SUMMARY

The present invention is directed to methods for enhancing or restoring the radiance of the complexion of skin, comprising applying to a part of the skin a cosmetic composition, formulated for topical application, comprising, as one of its cosmetically active agents, an effective amount of lipochroman-6.

### BRIEF DESCRIPTION OF THE DRAWINGS

FIG. 1 illustrates the action of lipochroman-6 on the Msr activity,

FIG. 2 illustrates the various steps (FIGS. 2a, 2b, 2c, 2d, 2e) of the image analysis that allows the level of oxidized proteins and the number of cells to be evaluated, and

FIG. 3 illustrates the effect of lipochroman-6 on the amount of oxidized proteins in the cells of the epidermis.

### DETAILED DESCRIPTION OF ILLUSTRATIVE EMBODIMENTS

The inventors of the present invention have demonstrated, surprisingly, that lipochroman-6 produces a reduction in the proportion of oxidized proteins in skin cells that are treated with this molecule, not only by stimulating the expression of genes coding for the enzymes MsrA and MsrB, but also by stimulating its activity of reversible repair of the oxidative damage, to give the proteins back their functionality.

The use of lipochroman-6 in these cosmetic compositions intended for skin application therefore allows the damage caused by the accumulation of these oxidized proteins, adversely affecting the proper functioning of the cell, to be limited, thereby contributing to an improvement in the complexion.

The improvement of the functioning of the cells of the epidermis that results from the use of lipochroman-6 leads to a regularization of cell renewal, and also of the differentiation of the keratinocytes migrating from the basal layer to the horny layer. This phenomenon of regularization of the cell renewal of the horny layer allows the surface of this horny layer to be made both more regular and smoother. Thus the incident light is better reflected by the surface (formed by the horny layer) of the skin, giving rise to the visual perception by the observer of a greater luminosity, of a more radiant skin complexion.

The invention thus firstly provides for the use of lipochroman-6 as an active agent in a cosmetic composition intended for application to at least part of the skin of the face, for the purpose of enhancing the radiance of the complexion.

The invention further provides for the use of lipochroman-6 as an active agent in a cosmetic composition intended for application to at least part of the skin of the body or the face, for enhancing and/or restoring the radiance of the complexion of the skin, especially where the alteration of the complexion is linked to skin ageing.

The purpose of this new use is especially to enhance and/or restore the radiance of the complexion of the skin of the face to which said composition is applied.

Another object of the invention is to provide a cosmetic care method intended for enhancing and/or restoring the radiance of the complexion of the skin.

Accordingly, according to a first essential feature, the present invention provides for the use in a cosmetic composition of lipochroman-6 as an active agent intended for enhancing and/or restoring the radiance of the complexion of the skin, especially of the skin of the face.

In one advantageous embodiment of the invention this active agent is contained in said composition in an amount sufficient to produce a reduction in the level of oxidized proteins in the cells of the skin following application of said composition to the skin.

In another advantageous embodiment of the invention this active agent is used in an amount sufficient to stimulate the expression of genes coding for the enzyme methionine sulphoxide reductase (Msr) and/or their oxidative damage repair activity, to give back the proteins their functionalities.

As is evident from the examples which follow, the active agent acts both on Msra and on Msrb, and especially on Msrb.

Lipochroman-6 is present within the cosmetic composition at a concentration of between 0.001% and 5% by weight of the composition, and preferably between 0.01% and 1% by weight.

According to a second essential feature, the invention relates to a method of cosmetic care of the skin, more particularly the skin of the face, which is intended to enhance and/or restore the radiance of the complexion. This method includes the application to the part of the skin in question of an effective amount of a cosmetic composition comprising lipochroman-6 as an agent intended for enhancing and/or restoring the radiance of the complexion of the skin.

Other features and advantages of the invention will emerge in the detailed description which follows, which is given in relation to the examples and the figures to which these examples refer.

It will be noted that Example 1 clearly demonstrates the effect of lipochroman-6 on the expression of the genes Msra and Msrb, and that Example 2 clearly shows the effect of this same lipochroman-6 on the activity of methionine sulphoxide reductase.

Example 3 in turn demonstrates the reduction in the level of oxidation of the proteins in the presence of lipochroman-6.

All of these effects of lipochroman-6 are confirmed by the remarkable properties of enhancement of the complexion that are obtained with the compositions exemplified in Examples 4 to 6.

Hence the new properties demonstrated by the inventors of the present invention result in the possibility of using lipochroman-6 in all the applications where enhancement of the radiance of the complexion of the skin, more particularly of the skin of the face, is desired.

### EXAMPLES

### Example 1

### Effect of Lipochroman-6 on the Expression of Genes Coding for the Proteins MsrA and MsrB

The effect of lipochroman-6 on the basal expression of genes coding for the proteins MsrA and MsrB is evaluated by the method of quantitative RT-PCR (real-time PCR; RT-Q-PCR).

The model selected is that of normal human epidermal keratinocytes (NHK) cultivated in vitro.

First of all a cytotoxicity test is carried out on keratinocytes.

The results of the MTT viability tests and the observation of the cell layers lead to selection of the non-cytotoxic concentrations for the remainder of the tests.

### Apparatus and Methods

### 1. Biological Model

Type: normal human epidermal keratinocytes (NHK)

Culture medium: SFM medium (Invitrogen)

Epidermal growth factor (EGF) 0.25 ng/ml and pituitary extract (PE) 25 μg/ml (EGF, PE, Invitrogen)

Gentamycin 25 μg/ml (Sigma)

Test medium: SFM medium without EGF and without PE (SFM-PE-EGF)

### 2. Test Products

Lipochroman-6: stock solution at 10 mg/ml in ethanol, diluted in test medium at concentrations of 0.01 mg/ml and 0.005 mg/ml.

### 3. Initial Cytotoxicity

96-well plates

cells/well: 15 000 NHK in SFM-PE-EGF medium

evaluation parameter: MTT hydrolysis, morphological observations

### 4. Cultures and Treatments

The keratinocytes are seeded and pre-cultured for 24 hours in complete SFM culture medium (24-well plates) and then placed in SFM-PE-EGF medium.

At confluence, the cells are treated or not (control) with the test product and are cultured for 24 h at 37° C. and 5% CO₂. Each treatment is carried out in triplicate. The culture supernatants are then harvested and the cell layers are rinsed with PBS solution (Invitrogen), and then 300 μl of tri-reagent (Sigma) are added. The plates are subsequently frozen at −80° C.

### 5. Quantitative RT-PCR (RT-Q-PCR)

The expression of the genes msra and msrb is evaluated by RT-Q-PCR on the basis of the messenger RNA (mRNA) extracted from the cell layers of each treatment. For each treatment, the three samples are combined into one prior to the extraction of the RNA.

5.1. Reverse Transcription

The main steps of the protocol are as follows:
Extraction of the total RNA using tri-reagent, according to the protocol of the supplier.
Removal of traces of potentially contaminating DNA by treatment with the DNA-free system (Ambion).
Implementation of the reverse transcription reaction in the presence of the primer oligo(dT) and the enzyme Superscript II (Gibco).

The PCR reactions are carried out with the aid of the following primers:

| Name of gene | Primer sequences |
|---|---|
| msra | TGGTTTTGCAGGAGGCTATAC (SEQ ID No: 1) |
| | GTAGATGGCCGAGCGGTACT (SEQ ID No: 2) |
| msrb | GCGACAGTCCACTCTTCAGTT (SEQ ID No: 3) |
| | CCCAGGTCCATCAGGAAACA (SEQ ID No: 4) |

The reference marker for the study is the following gene:

| Name of gene | GenBank No. |
|---|---|
| Liver glyceraldehyde 3-phosphate dehydrogenase | X01677 |
| (G3PDH) | |

5.2. Quantitative PCR

The PCR (polymerase chain reaction) reactions are carried out by quantitative PCR with the Light Cycler system (Roche Molecular Systems Inc.) according to the procedures recommended by the supplier. To perform correctly, this analytical system requires prior development of the conditions of analysis of the different primers. This system is formed of two main components: an optimized thermocycler, which allows extremely rapid heat transfers, and a fluorimeter, which continuously measures the fluorescence intensity incorporated in the DNA (detection at 521 nm). The reaction mixture (10 μl final) for each sample is as follows:
2.5 μl of cDNA diluted to 1/10th
primers of the different markers used
reaction mixture (Roche) containing the enzyme taq DNA polymerase, the marker SYBR Green I (a fluorophore which is integrated into the double-stranded DNA in the course of the elongation step) and MgCl₂.

5.3. Analysis of the Q-PCR

The incorporation of fluorescence into the amplified DNA is measured continuously during the PCR cycles. This system produces measurement curves of the fluorescence as a function of the PCR cycles, and hence allows evaluation of a relative expression value for each marker. The number of cycles is determined on the basis of the “exit” points of the fluorescence curves. For a given marker analysed, the later the exit of a sample (high number of cycles) the lower the initial number of copies of the mRNA. The relative expression (RE) value is expressed in arbitrary units (AU) in accordance with the following formula: (½^{number of cycles})×10⁶.

Results: Effects of lipchroman-6 on the Expression of the Genes msra and msrb

The results are presented in Tables 2 and 3.

TABLE 2
effects of treatment with lipochroman-6 on the relative expression of the gene MsrA.
Results related to the expression of the reference marker.
msra/G3PDH

| | | G3PDH | Mb | RE*G3PDH | RE*Mb | | % |
|---|---|---|---|---|---|---|---|
| Treatment | Conc. | Cycles | Cycles | (AU) | (AU) | Mb/G3PDH | Control |
| Control | — | 19.03 | 26.88 | 2.327 | 8.18 × 10⁻³ | 3.58 × 10⁻³ | 100 |
| | | 18.66 | 26.83 | | | | |
| | | 18.50 | 26.92 | | | | |
| Lipochroman-6 | 0.01 | 18.93 | 26.83 | 1.961 | 8.66 × 10⁻³ | 4.42 × 10⁻³ | 124 |
| | mg/ml | 18.99 | 26.79 | | | | |
| | | 18.96 | 26.73 | | | | |
| | 0.005 | 18.88 | 26.84 | | 8.251 × 10⁻³ | 4.14 × 10⁻³ | 116 |
| | mg/ml | 19.00 | 26.91 | 1.994 | | | |
| | | 18.93 | 26.81 | | | | |

RE*= Relative expression expressed in arbitrary units (AU), n = 3.

TABLE 3
effects of treatment with lipochroman-6 on the relative expression of the gene MsrB.
Results related to the expression of the reference marker.
msrb/G3PDH

| | | G3PDH | Mb | RE*G3PDH | RE*Mb | | % |
|---|---|---|---|---|---|---|---|
| Treatment | Conc. | Cycles | Cycles | (AU) | (AU) | Mb/G3PDH | Control |
| Control | — | 19.03 | 24.98 | 2.327 | 3.165 × 10⁻² | 1.38 × 10⁻² | 100 |
| | | 18.66 | 24.99 | | | | |
| | | 18.50 | 24.78 | | | | |
| Lipochroman-6 | 0.01 | 18.93 | 24.62 | 1.961 | 4.018 × 10⁻² | 2.05 × 10⁻² | 148 |
| | mg/ml | 18.99 | 24.60 | | | | |
| | | 18.96 | 24.49 | | | | |
| | 0.005 | 18.88 | 24.68 | 1.994 | 3.516 × 10⁻² | 1.76 × 10⁻² | 127 |
| | mg/ml | 19.00 | 24.86 | | | | |
| | | 18.93 | 24.75 | | | | |

RE*= Relative expression expressed in arbitrary units (AU), n = 3.

Conclusion: the results show that lipochroman-6 exhibits a greater inductive activity in relation to the expression of the gene msrb relative to the activity measured in relation to msra. This result conveys a specificity of action in the reduction of the R-form of methionine sulphoxide.

### Example 2

### Modulation of the Activity of Methionine Sulphoxide Reductase (Msr) by Lipochroman-6

### Apparatus and Methods

### 1. Media and Reagents

Keratinocyte Culture

Supplemented K-SFM:
keratinocyte-SFM with L-glutamine (Gibco® Invitrogen)
keratinocyte-SFM supplement (Gibco® Invitrogen)
phosphate buffer (PBS) ×10 pH 7.2 (Sigma-Aldrich)
penicillin-streptomycin (Sigma-Aldrich)
dimethyl sulphoxide DMSO (Sigma-Aldrich)
foetal bovine serum (FBS) (Gibco® Invitrogen)
0.25% trypsin-EDTA solution (Sigma-Aldrich)
Bio-Rad protein assay reagents (Biorad)
bovine serum albumin (Sigma-Aldrich)
DTT dithiothreitol (Amersham-Biosciences)
CelLytic™ (Sigma-Aldrich)
ethyl acetate (VWR)
Sigma-Fluor High (Sigma-Aldrich)
N-acetyl-(³H)-methionine-R,S sulphoxide substrate

(Amersham-Biosciences)

### 2. Cell Culture

The keratinocytes are obtained from grafts of skin originating from abdomens of healthy donors aged 47 years.

They are cultured in complete KSFM medium (KSFMc) at 37° C. and 5% CO₂ and then frozen in liquid nitrogen.

Thawing of the Cells

Before the cells are thawed, the temperature of the water bath is set to 37° C. and the supplemented KSFM culture medium (K-SFM-S) is preheated. The cryotube is removed from the liquid nitrogen and placed in the water bath at 37° C. until thawing takes place, and then is washed with 70% ethanol before opening. To remove the DMSO, the cells are withdrawn in a sterile procedure, placed in 10 ml of preheated medium, then resuspended by inversions.

Centrifugation is carried out at 200 g for 5 minutes at 4° C. The supernatant is drawn off and the cell pellet is taken up in 2 ml of K-SFM-S culture medium and placed in a T75 flask, where it is made up to 10 ml of culture medium.

The cells are placed in a moist incubator at 37° C. under a 5% CO₂ atmosphere. The culture medium is changed on the next day.

Cell Passage:

The cells which have achieved confluence are detached by the action of trypsin, and are counted and reseeded at 2.5×10⁶ cells per T75 culture flask.

Treatment of the Cells with Trypsin

The medium is drawn off and removed. The cells are washed with 2 ml of PBS buffer, which is drawn off and removed. Washing is repeated twice. 1 ml of preheated 0.25% trypsin-EDTA solution is added, diluted to ½ with PBS buffer. The flask is placed in the incubator at 5% CO₂ and 37° C. for 1 to 2 minutes. The action of the trypsin is stopped by addition of 2 ml of K-SFM-S, 50% (FBS) medium.

The cells are centrifuged at 200 g for 5 minutes at 4° C. The supernatant is drawn off and the cell pellet is taken up in 4 ml of culture medium. The cells are counted with a NucleoCounter from Chemometec (Bioblock) and seeded at 2.5×10⁶ cells per T75 flask in 10 ml of K-SFM-S culture medium. The cells are maintained in culture until passage P3. At passage P3, the cells are kept in culture overnight and used the next day for treatment with the actives.

3. Treatment of the Cells with Lipochroman-6

Preparation of the Lipochroman-6 Solutions

Lipochroman-6 powder (supplier: Lipotec) is first diluted in 20 μl of absolute ethanol. Then a stock solution at 10 mg/ml in the K-SFM-S medium is prepared.

Preparation of the Lipochroman-6 Dilution Series

An intermediate solution at 1 mg/ml (dilution 20 μl in 200 μl of K-SFM-S medium) is prepared from the lipochroman-6 stock solution.

The dilutions are carried out in the K-SFM-S medium on the basis of the intermediate solution at 1 mg/ml.

The lipochroman-6 concentration series is as follows:

| | Concentration |
|---|---|
| Solution No. | (mg/ml) |
| 0 | 0 |
| 1 | 0.0025 |
| 2 | 0.0050 |
| 3 | 0.0075 |
| 4 | 0.0100 |
| 5 | 0.0125 |
| 6 | 0.0150 |

Treatment of the Keratinocytes with Lipochroman-6

The medium is drawn off from the cell flasks (passage P3) and replaced with the medium containing the different dilutions of actives. After 24 hours of treatment, the cells are washed with PBS buffer and then treated with trypsin. The cells are finally counted with a NucleoCounter from Chemometec (Bioblock).

The treatment is carried out on 3.2 to 3.7×10⁷ cells per flask.

### 4. Protein Assay by the Bradford Method:

Preparation of protein extracts, cell lysis The cells are centrifuged at 900 g for 10 minutes at 4° C. The cell pellets are taken up in 1 ml of PBS buffer, placed in 1.5 ml Eppendorf tubes and then centrifuged at 450 g for 5 minutes at 4° C. The pellet is taken up in 150 μl of CelLytic™-M buffer. The cells are subsequently incubated for 15 minutes in a stirrer which is thermostated at 5° C. and are centrifuged for 15 minutes at 12 000 g. The protein supernatant is transferred to a new Eppendorf tube and kept at 5° C.

Protein Assay

Preparation of the Standard Series

Stock solution of bovine serum albumin (BSA) at 1 mg/ml

The series is prepared in accordance with the table below:

| Qty BSA (μg/tube) | BSA (μl) | H₂O (μl) |
|---|---|---|
| 0 | 0 | 100 |
| 2 | 2 | 98 |
| 4 | 4 | 96 |
| 6 | 6 | 94 |
| 8 | 8 | 92 |
| 10 | 10 | 90 |
| 12 | 12 | 88 |

Preparation of the Samples

The protein extracts are diluted to 1/10th and the assay is carried out on 10 μl made up to 100 μl of water. Each tube is admixed with 1 ml of Bio-Rad protein assay reagent (Bio-Rad) diluted ⅕th at the time of use in water. Following homogenization and 5 minutes of incubation at ambient temperature, the absorbance of the samples is measured on a spectrophotometer (Kontron Instrument) at a wavelength of 595 nm.

### 5. Measurement of the Msr Activity

Principle of the Assay

The Msr activity is measured on the basis of the protein extracts by using the substrate N-acetyl-(³H)-methionine-(R, S) sulphoxide in a reaction mixture of 30 μl containing Tris-HCl pH 7.5, 25 mM, MgCl₂ 10 mM, DTT 15 mM, at 37° C. The reaction is stopped by addition of 500 μl of 1 N HCl. The product of the reaction is extracted with 1.2 ml of ethyl acetate.

Protocol

The required quantity of proteins is withdrawn and made up with water to a reaction volume of 20.8 μl in a 2 ml Eppendorf tube stored in ice. 0.75 μl of Tris-HCl, pH 7.5, at 25 mM+3 μl of MgCl₂ at 10 mM+0.45 μl of DTT at 15 mM, followed by 5 μl of radioactive substrate diluted to 1/100th in water, are added. The tubes are agitated and incubated at 37° C. for 30 minutes to 2 hours, depending on the enzymatic kinetic conditions. The reaction is stopped with 500 μl of 1 N HCl. The product of the reaction is extracted with 1.2 ml of ethyl acetate and the tubes are agitated for 1 minute until an emulsion is obtained.

Following centrifugation at 15 000 g for 5 minutes at ambient temperature, 1 ml of the upper, organic phase is withdrawn and is counted in a scintillation vial and admixed with 2 ml of Sigma Fluor scintillant.

### 6. Determination of the Kinetics Conditions

The assays of Msr activities are carried out for each point in triplicate and are repeated twice. The conditions of the assay are as follows: 30 μg of proteins, incubation at 37° C. for 90 minutes.

### Results

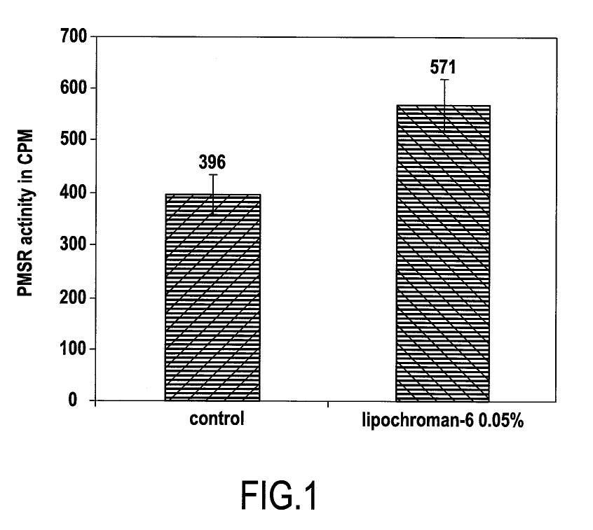

The action of lipochroman-6 on the Msr activity is shown in FIG. 1.

Conclusions:

The addition of lipochroman-6 to human keratinocytes in culture stimulates the Msr activity of these cells. At a dose of 0.05%, the activity is increased by 44%.

### Example 3

### Effect of Lipochroman-6 on the Oxidized Protein Content of Epidermal Cells

### Apparatus and Methods

### 1. Cell Culture

The cell manipulations are carried out in aseptic conditions under a laminar flow hood.

Normal human keratinocytes (NHK) isolated from harvesting of human skin carried out on two different donors (labelled KHN002 and KHN 9726 hereinafter) are cultured in T75 flasks in complete KSFM medium (KSFMc) (Invitrogen GIBCO) at 37° C. and 5% CO₂ and are seeded at 20 000 cells per well in 8-well Lab-Tek II culture systems (Nalge Nunc International).

### 2. Treatment

After 48 h of culture, the cells are treated with the treatment solutions (see below).

Preparation of the Lipochroman-6 Stock Solution

A 0.004% weight/volume lipochroman-6 solution is prepared (solution A).

This is done by dissolving 20 mg of lipochroman-6 powder (supplier: Lipotec) in 2 ml of ethanol (solution at 10 mg/ml). This solution is diluted (40 μl of the stock solution in 10 ml of KSFm medium) to give solution A.

Preparation of the Treatment Solutions

The treatment solutions are prepared by diluting solution A prepared above in the KSFMc medium, to give the correct percentage of lipochroman-6.

Treatments carried out on 3 wells per cell type:
control: KSFMc
solvent control: 0.1% solution of ethanol in KSFMc medium
0.001% lipochroman
0.002% lipochroman

### 3. Immunostaining of Oxidized Proteins

After 48 hours of treatment, the KSFMc is drawn off. The cells are rinsed with phosphate buffer (PBS) (PBS tablets, Invitrogen GIBCO), then fixed with formalin (formalin solution 10% neutral buffered, Sigma) for 10 minutes at ambient temperature. After rinsing with PBS, the culture chambers are filled with 0.1% triton solution (Triton X-100, Sigma) for 10 minutes, then rinsed with PBS.

The subsequently disassembled culture chambers and the slides are immersed in a solution of 2,4-dinitrophenylhydrazine (300 mg of DNPH (Fluka)) in an ethanol/sulphuric acid (98.5 ml/1.5 ml) mixture for 30 minutes.

The DNPH reacts with the carbonyl groups of the oxidized proteins in accordance with the following reaction:

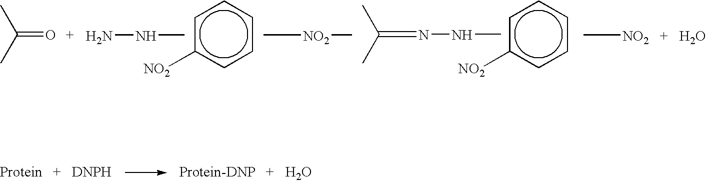

The slides are rinsed with PBS. The cells are subsequently covered with a 1% BSA/PBS solution (10 g/l) for 30 minutes at ambient temperature.

The dinitrophenyl (DNP) groups fixed to the oxidized proteins are recognized by a primary anti-DNP antibody.

For this purpose, in a first step, the slides are covered with a solution of primary antibody (monoclonal mouse anti-DNP antibody, Sigma), diluted to 1/500th in a 1% BSA/PBS solution, and are incubated for 60 minutes in an oven at ambient temperature and then rinsed with a 0.1% Tween-PBS solution (Tween20, Merck). In a second step, the cells are covered with a secondary antibody solution (Alexafluor 546 goat anti-mouse, Invitrogen) in a 1% BSA/PBS solution, and are incubated away from the light for 60 minutes. The slides are subsequently rinsed with PBS-Tween and then with PBS.

In a third step, a few drops of an aqueous mounting medium (fluorescent mounting medium, DAKO) are placed on the slides, which are subsequently stored at 4° C. in the dark.

### 4. Acquisition of Images in Confocal Microscopy

The photographs are made by confocal microscopy (Axioplan microscope from Zeiss and krypton-argon laser from BioRad) with the aid of the LaserSharp 2000 software (BioRad). For each condition, three photographs are made with the ×40 lens, according to the same acquisition parameters (gain, iris).

The excitation wavelength of the fluorochrome is 546 nm and the emission wavelength is 573 nm.

In practice, an excitation fluorescence at 546 nm is applied by the confocal laser to the cells. The fluorochrome Alexafluor 546, which represents the oxidized proteins, emits fluorescence at 573 nm, which is recovered by a specific filter and can be observed. The measured fluorescence is therefore directly proportional to the amount of oxidized proteins.

### 5. Image Analysis

The photographs are analysed with the aid of the Leica QWin image analysis software. A program allows the DNPH staining to be quantified in order to evaluate the level of oxidized proteins and the number of cells.

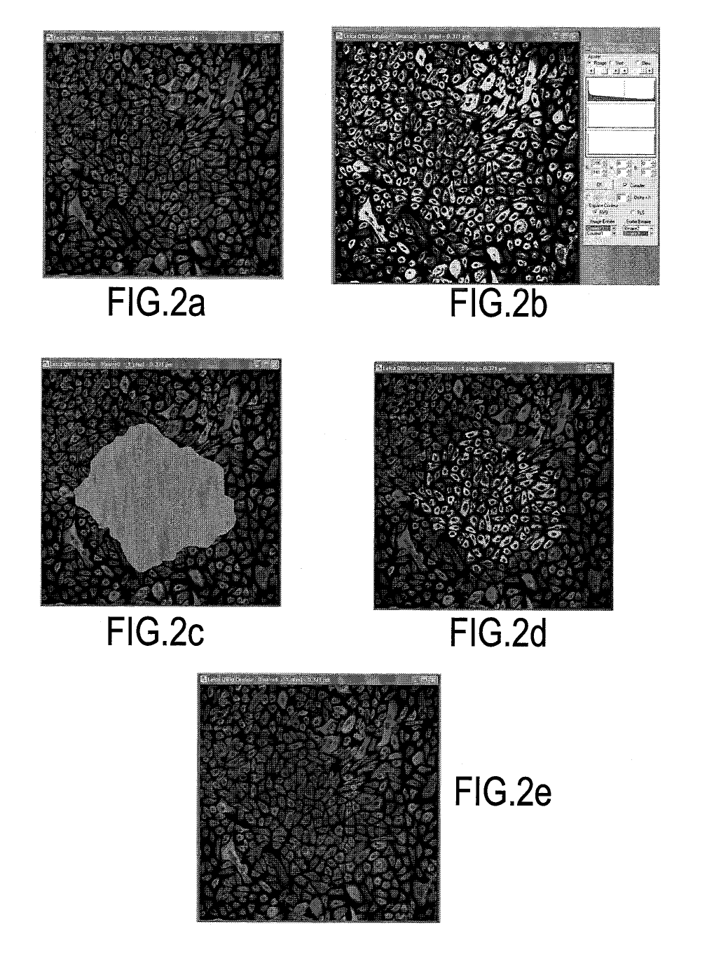

The program detects the fluorescent staining of the oxidized proteins. A detection zone which is as representative as possible (number, size, cell density, intensity of marking) is delimited. The fluorescence of the oxidized proteins is measured in the delimited zone, and the cells in the same zone are numbered by counting of the cell nuclei (see FIGS. 2a, 2b, 2c, 2d and 2e).

The process comprises, in order, the following steps:
opening of the image to be analysed (FIG. 2a)
detection of the staining of the oxidized proteins (FIG. 2b)
tracing of the detection zone (FIG. 2c) and subtraction of the stained zones (FIG. 2d)
checking-off of the cells (FIG. 2e).

### Results

The effect of lipochroman-6 on the oxidation of proteins is measured on the two types of normal human keratinocytes (NHK). This is done by estimating the surface area of staining of the oxidized proteins, the surface area of the measurement zone and the number of cells in the measurement surface area, using an image processing system.

### 1. Statistical Methods

Missing Data, Outliers

The percentages are calculated on the basis of those persons responding (n=6) except for cases of missing values or absence of response, where the base is indicated by “_{/n=5}”. For the paired tests, if there is a missing value for one subject, that subject is removed from the analysis. The Dixon test is used to determine the outliers.

Degree of Significance of the Statistical Tests

Statistical analysis is carried out with an error risk α=5%.

p indicates the significance of the test:
if p≦0.05 [symbol] S (significant), the value of p is reported in brackets.
if 0.05≦p≦0.10 [symbol] Slim (borderline significant), the value of p is reported in brackets.
if p>0.10 [symbol] NS (not significant), the value of p is not reported.

Analysis of the Qualitative Data

Distribution sorting of the qualitative questions involves recording the total numbers and then calculating the frequencies of the different possible responses (expressed as a percentage). The χ² test is used to compare the percentages.

Analysis of the Quantitative Data

First of all a descriptive analysis is conducted. Analysis of variance (ANOVA) is used to compare the means of the modalities of a factor. If the Student test is used, it is indicated by a ‘T’.

Software

Statgraphics Plus Centurion package and Uniwin 6.0 under Windows NT.

2. Effect of Treatment with Lipochroman-6 on the Oxidized Protein Content of Skin Cells.

Unilateral tests are used to calculate the significances, except for the solvent control, for which a bilateral test is used: control/solvent control.

Proportion of oxidized proteins/cells

| | | | | Reduction |
|---|---|---|---|---|
| | | | | %/control |
| | KHN 0002 | KHN 9726 | Mean | excipient |
| Control | 102.1 | 102.9 | 102.5 | |
| Control excipient | 93.6 | 110.2 | 101.9 | 0.00% |
| | NS | NS | NS | |
| Lipochroman-6 | 76.3 | 88 | 82.1 | −19.43% |
| 0.001% | S (<0.01) | S (<0.01) | S (<0.01) | |
| Lipochroman-6 | 60.9 | 43 | 52 | −48.97% |
| 0.002% | S (=0.01) | S (<0.001) | S (<0.001) | |

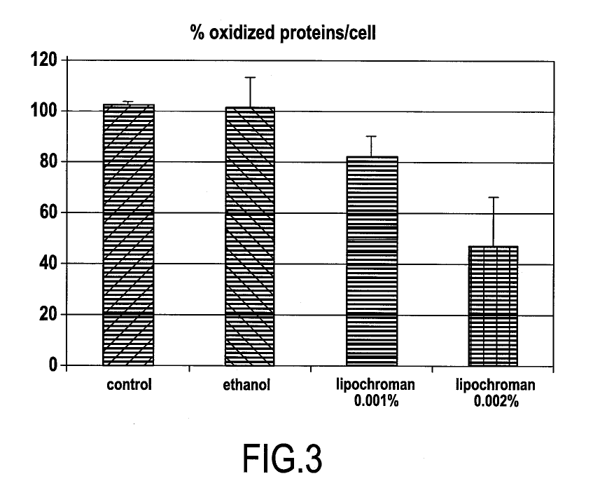

The proportions of oxidized proteins in the cells treated with the lipochroman-6 solutions are shown in FIG. 3.

Conclusion: a significant effect of the lipochroman-6 on the oxidation of the proteins is observed for each test. The greater the concentration of lipochroman-6, the lower the surface area of marked oxidized proteins. Thus the proportion of oxidized proteins reduces significantly after treatment with one or other of the lipochroman-6 solutions (−19.43% and −48.97% respectively, for the 0.001% or 0.002% solutions of lipochroman-6).

The table below distinguishes the significance of the efficacy of the treatments with lipochroman-6 relative to the controls.

| | | DNP marking/ | |
|---|---|---|---|
| | Surface area | surface area | DNP marking/ |
| | DNP marking | measured | No. of cells |
| Control (1) | 5341 D | 36.4% E | 102.5 C |
| Ethanol (2) | 5247 D | 38.1% E | 101.9 C |
| Lipochroman 0.001% | 3776 C | 28.7% D | 82.1 B |
| Lipochroman 0.002% | 2415 B | 18.4% B | 52.0 A |
| Significance | S (<0.001) | S (<0.001) | S (<0.001) |

Effect on the surface area of oxidized proteins, from smallest to largest.
If letter is the same = treatment of comparable efficacy.
If letters are different = treatment of significantly different efficacy.

### Example 4

### Anti-Wrinkle Day Cream to Boost the Luminosity of the Complexion

The composition is an oil-in-water emulsion comprising lipochroman-6 (% expressed by weight relative to the weight of the composition):

| | |
|---|---|
| Lipochroman-6 | 0.05 |
| Centella asiatica extract | 0.5 |
| Steareth-21 | 2.5 |
| Glyceryl stearate | 1.1 |
| Stearyl alcohol | 5 |
| Glycerol tricaprate/caprylate | 11.5 |
| Butylene glycol | 3 |
| Glycerol | 2 |
| Preservative | 0.5 |
| Perfume concentrate | 0.5 |
| UV filter | 7.5 |
| Water | qs 100 |

The cream is applied to the face, preferably in the morning.

### Example 5

### “Radiance Shot” Lotion Comprising Lipochroman-6

The cosmetic composition is a lotion (% expressed by weight relative to the weight of the composition):

| | |
|---|---|
| Lipochroman-6 | 0.05 |
| Butylene glycol | 3 |
| EDTA | 0.1 |
| Solubilizer | 1 |
| Perfume concentrate | 0.3 |
| Ethanol | 5 |
| UV filter | 0.1 |
| Water | qs 100 |

The lotion, when applied to the face, for example at the end of the day for the evening, restores the radiance of the face.

### Example 6

### Make-Up Powder Comprising Lipochroman-6

The cosmetic composition is a compact powder (% expressed by weight relative to the weight of the composition):

| | |
|---|---|
| Lipochroman-6 | 0.05 |
| Moisturizing active (glycerol) | 2.4 |
| Cohesion agents | 3.5 |
| UV filters | 18 |
| Texture agents | 49 |
| Binder | 16 |
| Perfume concentrate | 0.1 |
| Preservatives | 0.6 |
| Pigments | 6 |
| Excipient (talc, mica) | qs 100 |

The powder, when applied to the face, imparts a coloured effect and restores the radiance of the face.

What is claimed:

1. A method for enhancing or restoring the radiance of the complexion of skin, comprising:
applying to a part of the skin a cosmetic composition, formulated for topical application, comprising an effective amount of lipochroman-6.

2. The method according to claim 1, wherein the lipochroman-6 is present in an amount effective to produce a reduction in the proportion of oxidized proteins in the cells of the skin.

3. The method according to claim 1, wherein the lipochroman-6 is present in an amount effective to stimulate the expression of genes coding for methionine sulphoxide reductase (Msr) and/or the oxidative damage repair activity of methionine sulphoxide reductase.

4. The method according to claim 1, wherein the lipochroman-6 concentration of said composition is between 0.001% and 5% by weight of the composition.

5. The method according to claim 4, wherein the lipochroman-6 concentration of said composition is between 0.01% and 1% by weight.
